(19) United States
(12) Patent Application Publication
(10) Pub. No.: US 2008/0181955 A1
(43) Pub. Date: Jul. 31, 2008

(54) Natural dietary supplement and method for enhancing human libido

(76) Inventor: Lawrence X. Webb, Winston-Salem, NC (US)

Correspondence Address: CHRISTOPHER C. DREMANN, P.C., 18 MALLARD COURT, GRANITE FALLS, NC, 28630, US

(21) Appl. No.: 11/699,688

(22) Filed: Jan. 30, 2007

(51) Int. Cl. A61K 31/05; A61K 36/899; A61K 38/43; A61K 38/46

(52) U.S. Cl. 424/489; 424/750; 424/94.1; 424/94.6; 514/733

(57) ABSTRACT

A dietary supplement includes resveratrol in a therapeutically effective amount to permit release of nitric oxide (NO) during sexual stimulation and at least one polyphenol in a therapeutically effective amount to inhibit formation of type 5 phosphodiesterase (PDE-5). The dietary supplement is a dry powder derived from Muscadine grapes that are pressed and then stored at a temperature greater than 32° F. (0° C.) and less than 60° F. (15° C.) for at least 14 days. The pressed and stored grapes are dried and then ground into the dry powder. The concentration of the resveratrol is between 0.01 mg and 100 mg per 100 g of dry powder and the concentration of the polyphenol is between 0.04 mg and 400 mg per 100 g. A method for enhancing human libido includes providing the dry powder and consuming at least 50 mg of the dry powder daily for a period of at least 21 days.

### FIELD OF THE INVENTION

This invention relates to a dietary supplement and to a method for enhancing human libido. More particularly, the invention is a natural dietary supplement that inhibits the production of type 5 phosphodiesterase (PDE-5) during sexual stimulation. The dietary supplement is produced using an improved method for processing Muscadine grapes into a dry powder having a high concentration of polyphenolic compounds, and especially resveratrol.

### BACKGROUND OF THE INVENTION

It is widely accepted that human sexual desire, commonly known as libido, is affected by both psychological and physiological factors. Physiologically, sexual stimulation and arousal are accompanied by an increase in blood flow to the genitalia, and particularly to the male penis and to the female vulva. Psychologically, an often cited reason for reduced libido in men is the inability to develop or maintain an erection of the penis for satisfactory sexual intercourse. The inability to develop or maintain an erection is a medical condition clinically known as erectile dysfunction (ED) and commonly referred to as impotence. The causes of ED may likewise be psychological or physiological. Psychological impotence is often overcome by the placebo effect of almost anything that the patient believes will help. Physiological impotence is most often treated pharmaceutically with one of the three leading ED drugs; namely, sildenafil (commercially known as Viagra® and available from Pfizer Inc. of New York, N.Y.), vardenafil (commercially known as Levitra® and available from Bayer Aktiengesellschaft of the Federal Republic of Germany), or tadalafil (commercially known as Cialis® and available from Lilly ICOS LLC of Wilmington, Del.). All three of these drugs are taken orally within a relatively short period of time prior to planned sexual activity. All three act to increase blood flow within the penis in response to sexual stimulation by inhibiting formation of type 5 phosphodiesterase (PDE-5). It has been clinically proven that an erection of the male penis can be produced by a non-specific vasodilator acting as an alpha-blocking agent that causes corporal smooth muscle relaxation and inflow of blood to the corpus cavernosum. The physiological mechanism of an erection of the penis includes release of nitric oxide (NO) in the corpus cavernosum during sexual stimulation. The NO released in the corpus cavernosum activates the enzyme guanylate cyclase, which results in increased levels of cyclic guanosine monophosphate (cGMP), thereby producing smooth muscle relaxation in the corpus cavernosum and permitting inflow of blood (i.e., vasodilatation). ED drugs have no direct smooth muscle relaxing effect on the corpus cavernosum in isolation (i.e., in the absence of sexual stimulation). However, the enzyme activating effect of the NO released during sexual stimulation is enhanced by ED drugs since they inhibit the formation of PDE-5, which is primarily responsible for the degradation of cGMP in the corpus cavernosum. In short, when sexual stimulation causes local release of NO, the inhibition of PDE-5 by an ED drug results in increased levels of cGMP, which in turn allows smooth muscle relaxation and inflow of blood to the corpus cavernosum of the penis.

While the known ED drugs are generally effective for their intended purpose, use of such drugs is not without risk or inconvenience. A percentage of patients suffer from known side effects and the long term side effects of ED drugs are as yet unknown. In particular, hepatic impairment and severe renal impairment have been shown to be associated with increased plasma levels of sildenafil (Viagra®). In addition, recommended doses of sildenafil are known to temporarily produce a decrease in supine blood pressure and impairment of blue/green color discrimination. Although less severe, the use of ED drugs can result in headache, flushing, stomach upset, nasal stuffiness, diarrhea, dizziness, painful urination and skin rash. As a result, patients having certain chronic medical conditions, for example diabetes or high blood pressure, are advised not to take ED drugs. Furthermore, ED drugs should not be taken with an M.A.O. inhibitor and concomitant nitrates should be avoided since a potential for serious hypotension exists in the event that an organic nitrate is combined with a PDE-5 inhibitor. But perhaps a greater disadvantage for more patients is the inconvenience associated with the use of ED drugs. Sildenafil (Viagra®) is rapidly absorbed with maximum plasma concentrations reached within about 60 minutes, but has a half-life of only about 4 hours. Tadalafil (Cialis®) is less rapidly absorbed and has a longer period of effectiveness with a half-life of about 17.5 hours. Regardless, a patient must take the drug within a prescribed time in advance of planned sexual activity and enjoys only a limited “window of opportunity” for maximum effectiveness. The relatively high cost of ED drugs typically prevents a patient from taking the drug on a more frequent basis when only the likelihood or possibility of sexual activity exists. Finally, while sexual dysfunction drugs currently available are effective for treating ED in males, they appear to have no apparent therapeutic benefit for females.

Numerous commercially available dietary supplements and herbal supplements claim to increase sexual desire in males, females, or both. For example, U.S. Pat. No. 6,638,541 issued Oct. 28, 2003, to Hudson et al. discloses a sexual desire and performance enhancement including at least partially defatted meal from a plant source containing protein-bound tryptophan. A limited number of subjects participating in a selected sleep study who ingested a high protein food composition rich in protein-bound tryptophan reported an increase in sexual desire and performance. Tryptophan is an amino acid found in numerous naturally occurring plant proteins and is commonly utilized to treat insomnia and as an adjunct in the treatment of many psychiatric disorders. After absorption and under appropriate conditions, tryptophan is transported into the brain and across the blood brain barrier (BBB) where it becomes available for metabolism into serotonin. For this reason, the effects of tryptophan are not always consistent or predictable. Furthermore, tryptophan is not known to provide any other significant benefits to general health or overall well being. Finally, the enhancement disclosed in the Hudson et al. patent is derived from a plant source naturally containing protein-bound tryptophan, and particularly squash seeds, that is at least partially defatted to concentrate the amount of protein-bound tryptophan. However, there is no known method for processing tryptophan that results in a concentration of the protein-bound tryptophan up to several times that which occurs naturally in squash seeds.

Accordingly, what is needed is a dietary supplement and a method for enhancing human libido utilizing the dietary supplement. It is desirable that the dietary supplement and method provide the benefits of the common sexual dysfunction drugs, such as ED drugs, without the accompanying disadvantages. In particular, it is desirable that the dietary supplement be natural, and thereby free from any known or as yet unknown side effects. In addition, it is desirable that the dietary supplement and method be continuous acting, so to be convenient and thereby not require sexual activity to be planned, while remaining cost effective to use. It is also desirable that the dietary supplement and method be effective to enhance libido in both males and females. Finally, it is desirable that the natural dietary supplement be processed in a manner that results in a concentration of the active ingredient up to several times that which occurs naturally in the substance.

### SUMMARY OF THE INVENTION

In one aspect, the invention is embodied by a dietary supplement including resveratrol in an amount sufficient to permit release of nitric oxide (NO) during sexual stimulation and at least one polyphenol in an amount sufficient to inhibit formation of type 5 phosphodiesterase (PDE-5). The dietary supplement is a dry powder derived from grapes that are pressed to remove at least about fifty percent (50%) of the moisture content of the grapes and then stored at a temperature greater than about 32° F. (0° C.) and less than about 60° F. (15° C.). The pressed grapes are stored for at least about 14 days before further processing, and more preferably, are stored for at least about 90 days before further processing. The pressed and stored grapes are dried and then ground into the dry powder. The dry powder has a concentration of the resveratrol that is greater than at least three times the concentration of the resveratrol when the grapes are not stored for at least 14 days at a temperature greater than about 32° F. (0° C.) and less than about 60° F. (15° C.) before the pressed and stored grapes are dried and then ground. In particular, the concentration of the resveratrol is between about 0.01 mg and about 100 mg per 100 g of the dry powder and the concentration of the at least one polyphenol is between about 0.04 mg and about 400 mg per 100 g of the dry powder. In one embodiment, the pressed and stored grapes are dried using high air flow convection without the addition of heat in excess of about 120° F. (50° C.) and the dried grapes are ground at a temperature less than about 77° F. (25° C.). In a preferred embodiment, the grapes are Muscadine grapes and the pressed grapes consist essentially of Muscadine grape skins.

In another aspect, the invention is embodied by a dietary supplement having the effect of enhancing human libido. The dietary supplement includes resveratrol in a therapeutically effective amount and at least one polyphenol in a therapeutically effective amount. The therapeutically effective amount of resveratrol and the therapeutically effective amount of the at least one polyphenol are derived from grapes that are pressed to remove at least about fifty percent (50%) of the moisture content of the grapes and then stored at a temperature greater than about 32° F. (0° C.) and less than about 60° F. (15° C.). In one embodiment, the dietary supplement further includes rice hulls mixed with the grapes in a ratio of grapes to rice hulls by weight of at least about 10:1. In another embodiment, the dietary supplement further includes at least one active enzyme selected from the group consisting of pectinase and amylase types. The pressed grapes are stored for at least about 14 days before further processing, and more preferably, are stored for at least about 90 days before further processing. The pressed and stored grapes are dried and then ground into a dry powder. The dry powder has a concentration of the resveratrol that is greater than at least three times the concentration of the resveratrol when the grapes are not stored for at least 14 days at a temperature greater than about 32° F. (0° C.) and less than about 60° F. (15° C.) before the pressed and stored grapes are dried and then ground. In particular, the concentration of the resveratrol is between about 0.01 mg and about 100 mg per 100 g of the dry powder and the concentration of the at least one polyphenol is between about 0.04 mg and about 400 mg per 100 g of the dry powder. In one embodiment, the pressed and stored grapes are dried using high air flow convection without the addition of heat in excess of about 120° F. (50° C.) and the dried grapes are ground at a temperature less than about 77° F. (25° C.). In a preferred embodiment, the grapes are Muscadine grapes and the pressed grapes consist essentially of Muscadine grape skins.

In yet another aspect, the invention is embodied by a method for enhancing human libido. The method includes providing a dry powder having a therapeutically effective amount of resveratrol to release nitric oxide (NO) within the corpus cavernosum of the penis during sexual stimulation and a therapeutically effective amount of a polyphenol to inhibit formation of type 5 phosphodiesterase (PDE-5). The method further includes consuming at least about 50 mg of the dry powder at least about once a day for a period of at least about 14 days. The dry powder is derived from grapes that are pressed to remove at least about fifty percent (50%) of the moisture content of the grapes and then stored for at least about 14 days at a temperature greater than about 32° F. (0° C.) and less than about 60° F. (15° C.). The pressed and stored grapes are dried to remove essentially all of the moisture content of the grapes and then ground into the dry powder. In one embodiment, the pressed and stored grapes are dried using high air flow convection without the addition of heat in excess of about 120° F. (50° C.). In another embodiment, the grapes are pressed and ground at a temperature less than about 77° F. (25° C.). The grapes may be mixed with rice hulls in a ratio of grapes to rice hulls by weight of at least about 10:1. At least one active enzyme selected from the group consisting of pectinase and amylase types may also be added to the mixture. The dry powder has a concentration of the resveratrol that is greater than at least 3 times the concentration of the resveratrol when the grapes are not stored for at least 14 days at a temperature greater than about 32° F. (0° C.) and less than about 60° F. (15° C.) before the pressed and stored grapes are dried and ground. In particular, the concentration of the resveratrol is between about 0.01 mg and about 100 mg per 100 g of the dry powder and the concentration of the polyphenol is between about 0.04 mg and about 400 mg per 100 g of the dry powder.

### BRIEF DESCRIPTION OF THE DRAWINGS

These and other features, aspects and advantages of the present invention are better understood when the following detailed description of the invention is read with reference to the accompanying drawings, in which like reference numerals indicate like parts. The drawing figures illustrate various exemplary embodiments of the invention described in greater detail below. Therefore, the drawing figures should not be construed to limit the scope of the invention in any manner to the particular examples shown and described herein.

FIG. 1 is a schematic diagram illustrating an exemplary apparatus for processing organic plant matter, and in particular grapes, into a dry powder according to the present invention.

FIG. 2 is a flowchart illustrating the steps of an exemplary method for processing Muscadine grapes and rice hulls into a dry powder using the apparatus of FIG. 1.

FIG. 3 is a table listing the ranges of concentrations by weight of various polyphenolic compounds per 100 grams (100 g) of a dry powder processed from a mixture of Muscadine grapes and rice hulls using the apparatus of FIG. 1 in accordance with the method of FIG. 2.

### DETAILED DESCRIPTION OF THE INVENTION

Organic plant matter is known to contain numerous biologically active (also referred to herein as “bioactive”) components, such as vitamins, minerals, nutrients and natural antioxidants, that provide significant nutritional, health and therapeutic benefits. For example, many natural substances of plant matter origin contain polyphenolic compounds that exert anti-tumor and antioxidant activity, block the formation of free radicals, neutralize existing free radicals, reduce oxidative enzymes and decrease the concentration of lipoperoxides in plasma. These effects are principally attributed to the polyphenolic compounds, including resveratrol, naturally occurring in certain vegetables, fruits and nuts (e.g., peanuts), and especially in grapes and grape by-products (e.g., grape vines, stems, leaves, roots, seeds, etc.). Resveratrol is a natural phytoalexin known to inhibit tissue factor expression in vascular cells, and therefore, is believed to be beneficial in preventing heart disease and certain cancers, as well as blood disorders, inflammatory conditions such as arthritis, and other maladies. Grapes and grape by-products contain several bioactive polyphenolic compounds, including flavonoids (e.g., catechins and oligomers thereof, flavonols and anthocyanidols; proanthocyanidins; quercitins; etc.) and non-flavonoids (e.g., stilbene derivatives, such as resveratrol). It is the non-flavonoid resveratrol, however, that is regarded as being most responsible for the beneficial effects on the human cardiovascular system attributed to grape-derived products, such as wine, grape juice, grape powder, grape seed powder and grape seed oil. Although the underlying mechanisms are not entirely understood, the chemo-protective actions of resveratrol are believed to be related at least in part to its free radical negating and anti-inflammatory effect.

Numerous methods exist for extracting the beneficial bioactive components from organic plant matter. Typically, the organic plant matter is first crushed, squeezed or pressed to extract the juice. The natural pectin in the juice is then removed and the juice subjected to a high heat drying process to remove most of the liquid. Ideally, the high heat drying process results in a dry powder that can be incorporated into various food additives, nutriments, dietary supplements, nutraceuticals, pharmaceuticals, and health and beauty products, such as cosmetics. Such pressing and high heat drying processes are utilized for extracting polyphenolic compounds from grapes and grape by-products. These processing methods, however, generally suffer from inherent limitations that are particularly prevalent when utilized to process grapes and grape by-products. First and foremost, the juice contains only a fraction of the beneficial polyphenolic compounds available from the grapes and grape by-products. As a result, vast quantities of the juice must be processed in order to obtain any appreciable amount of the polyphenolic compounds. Furthermore, the solid residue that remains after extracting the juice, generally referred to as the “pomace,” and more specifically referred to as the “marc” with reference to fermented grapes used in wine production, is typically discarded and not utilized for any commercial purpose. Recently though, it has become widely recognized that the highest concentration of the beneficial bioactive components, and in particular polyphenolic compounds including antioxidants such as resveratrol, reside in the husks, skins and seeds of the grapes. It is these components that constitute the bulk of the pomace, as well as the marc, that is discarded in the known extraction methods.

Another problem encountered with the known extraction methods for organic plant matter, including grapes and grape by-products, is that high heat processing significantly degrades the efficacy of most bioactive components. In particular, high heat drying in a conventional drying and/or fragmenting apparatus causes the natural sugars present in the organic plant matter to caramelize and coagulate into a viscous mass. Consequently, vegetables and fruits containing high concentrations of natural sugars can clog the drying apparatus when subjected to elevated temperatures requiring the drying apparatus to be cleaned repetitively and resulting in inefficient and uneconomical processing of the dry powder. U.S. Pat. No. 6,190,716 to Galbreath, Jr. discloses a method for preparing a natural product, such as a food supplement containing resveratrol derived from Muscadine grapes. The grapes are first crushed and de-juiced. The pulp of the crushed and de-juiced Muscadine grapes is then broken down by heating the pomace and with optional enzymatic action. The broken down pulp is then extracted from the pomace and the skins and seeds of the extracted pulp are collected into net bags. The net bags are then placed into a conventional rotary drum dryer at an elevated temperature of about one hundred eighty degrees Fahrenheit (180° F.) and the contents dried to less than about one percent (1%) moisture. The dried material is next gathered into plastic freezer bags and stored in food grade containers at slightly less than room temperature (between about 60° F. and 70° F.). After a predetermined time, the dried material is ground into a fine, dry powder that may be encapsulated into gelatin capsules or combined with other food products.

U.S. Pat. No. 4,950,491 to Varga discloses an alternative process for preparing dry powdered fruit from whole fruit or from the by-products of fruit processing, and in particular, the husk residue (i.e., pomace) of fruit processing. The fruit or fruit by-products are cleaned, cut-up and then dried in a microwave oven at a maximum temperature of about sixty-five degrees Celsius (65° C.). If necessary, the microwave dried fruit or fruit by-products may be further dried using an infrared radiator, such as a lamp, to gently reduce the moisture content at the surface of the fruit matter. Regardless, the dried fruit matter is then ground in a suitable grinding apparatus to a particle size ranging between fine powder and coarse grist. If desired, the ground fruit matter can be fractionated, for example in a cyclone, to obtain different fractions of matter ranging in size between mealy-fine and granular crumbly. The gentle microwave drying process increases the efficacy of the bioactive components. However, a substantial amount of time and energy is required to process the fruit matter. Accordingly, the process is not efficient for the production of large quantities of a dry powder.

U.S. Pat. No. 6,479,081 to Feries discloses a method for obtaining grape pomace and grape seed tannin comprising a first step of extracting a crude tannin fraction by solid-liquid extraction from a mixture of grape marc and/or seed in an aqueous solvent of sulphited water (H₂O+SO₂). In a second step, the aqueous solvent is then removed from the extract to produce a concentrate of the crude tannin fraction. In a third step, the crude tannin fraction is then purified by liquid-liquid extraction through evaporation of the solvent to solubilize the tannin, selective adsorption of the polyphenolic compounds of the tannin on resin, and subsequent filtration on activated charcoal. Advantageously, the crude tannin fraction is extracted in the first step at a temperature of at most 25° C., and preferably between about 15° C. and 25° C. Preferably, the aqueous solvent is removed in the second step by evaporation under vacuum at a temperature of at most 75° C. The resulting concentrate is immediately cooled to a temperature of at most 30° C. Preferably, the crude tannin fraction adsorbed on resin in the third step is filtered by selective diafiltration at a temperature of at most 25° C. Even if the elevated temperature of the drying process does not caramelize the natural sugars of the organic plant matter, and regardless whether the extraction method includes process steps that subject the organic plant matter to excessive temperature, the plant material nevertheless may clump together within a conventional drying apparatus. In particular, the organic plant matter may cling to the inner surface of the wall of the drying apparatus, thereby subjecting the material to friction-induced heating and prolonged exposure to elevated temperature. Furthermore, the organic plant matter residue clinging to the inner surface of the wall of the drying apparatus is not available to be fragmented into a dry powder. The prolonged exposure to heat can significantly reduce the efficacy of the bioactive components for the reasons previously discussed, while the reduced fragmentation of the organic plant matter limits the efficiency of the extraction method. Accordingly, a conventional drying apparatus that simultaneously removes moisture and fragments the organic plant matter, such as a cyclonic dryer, further limits the efficiency of processing a dry powder and its constituent polyphenolic compounds, including resveratrol.

An improved method for processing organic plant matter into a dry powder comprising a high concentration of bioactive components, and in particular polyphenolic compounds, including resveratrol, is described in U.S. patent application Ser. No. 11/442,794 to Dalton filed on May 30, 2006, and published on Dec. 14, 2006, as United States Patent Application Publication No. 2006/0277887, the content of which is expressly incorporated herein in its entirety. The method includes pressing and/or drying organic plant matter to remove the bulk of the moisture and then grinding the pressed/dried plant matter to produce a dry powder that can be utilized alone or in combination with other constituents as a food additive, nutriment, dietary supplement, nutraceutical, pharmaceutical or a component of a cosmetic product that provides a nutritional, health and/or therapeutic benefit. Importantly, the de-juiced organic plant matter is stored at a temperature only slightly above 32° F. (0° C.) in order to promote the continued metabolic synthesis of the beneficial bioactive components, thereby further increasing the resulting concentration of the polyphenolic compounds that provide the nutritional, health and/or therapeutic benefits. As discussed in the Dalton patent application incorporated herein the concentration of resveratrol measured in mg/g of the resulting dry powder experienced an average over 9-fold increase when stored at slightly above freezing temperature for greater than 3 months (90 days), and experienced an average almost 4-fold increase when stored at slightly above freezing temperature for more than 14 days, but less than 3 months (90 days).

Preferably, both the pressing and grinding steps are likewise performed at a processing temperature that is sufficiently below ambient room temperature, for example less than about 77° F. (25° C.) and preferably between about 32° F. (0° C.) and about 60° F. (15° C.), to preserve the efficacy of the polyphenolic compounds, including resveratrol. An active enzyme may be added to the organic plant matter to facilitate breaking down the plant matter prior to pressing or to direct the metabolic activity of the plant matter during cold storage to enhance the production of polyphenolic compounds, including resveratrol. By directing metabolism, the active enzyme makes the polyphenolic compounds more bio-available. In certain instances, the organic plant matter is first pressed and then separated into two or more subcomponents (e.g., juice, pomace and seeds) that produce both a synergistic effect when processed together, as well as a nutritional, health and/or therapeutic benefit when processed on their own. The separated components may then be individually dried and ground into a dry powder, or may be further processed in any conventional manner, such as subsequent pressing to extract an oil product. Regardless, the resulting dry powder, oil and juice products are useful alone or in combination with other constituents that can be incorporated into various food additives, nutriments, dietary supplements, nutraceuticals, pharmaceuticals and cosmetic products, such as health or beauty aids, having a high concentration of bioactive components, and in particular natural antioxidants, such as the polyphenolic compounds, including resveratrol, that provide a significant nutritional, health and/or therapeutic benefit.

In a preferred embodiment, the organic plant matter comprises grapes and grape by-products. As used herein, the term “grape by-products” refers to components of the grape plant other than the grapes themselves, which are conventionally used to produce commercial grape-derived beverages, such as wines and grape juices, and vinegars. In particular, the grape by-products may include grape vines, stems, leaves, roots and seeds. If necessary, the grapes and grape by-products are first mixed together and the resulting mass is then pressed to remove the bulk of the liquid grape juice. Preferably, at least about fifty percent (50%) and more preferably at least about seventy percent (70%) of the moisture is removed from the mixture of grapes and grape by-products. An active enzyme or combination of enzymes may be added to the mixture of grapes and grape by-products before or after pressing. Preferably, the active enzyme is added prior to pressing the mixture for the purpose of softening the skins of the grapes and thereby facilitating the break down of the grapes and grape by-products. However, enzymatic action may also direct the metabolic activity of the pressed mixture (i.e., pomace) during cold storage to enhance the production of polyphenolic compounds, including resveratrol. Any suitable enzyme may be employed for either or a different purpose. For example, active enzymes of the pectinase and amylase types may be utilized to cleave different parts of the grapes, and in particular the skins, so as to render the grapes softer and easier to mechanically break apart during pressing and subsequent processing steps. Regardless, the pressed mixture (i.e., the de-juiced pomace and grape by-products) is then stored at a temperature only slightly above 32° F. (0° C.) for an extended period of time in order to promote continued metabolic synthesis and thereby increase the concentration of the polyphenolic compounds, including resveratrol in the resulting dry powder, oil and juice products. At the same time, “cold storage” of the pressed mixture also safeguards the accumulated antioxidant polyphenolic compounds against thermal or oxidative degradation.

Preferably, only fresh grapes and grape by-products are pressed. In the event it is necessary to store the grapes and grape by-products for an extended period of time prior to pressing, they may likewise be stored, for example in a freezer, at a temperature significantly below ambient temperature. The pressed mixture remains in the cold storage for at least about 14 days, and more preferably at least about 3 months (90 days). During that time, an additional amount of moisture naturally evaporates from the mixture so that about as much as eighty percent (80%) of the moisture of the original mixture is removed. Following cold storage, the pressed mixture is pressed a second time to further reduce the amount of moisture remaining in the mixture. The second pressing produces a juice residue comprising a high concentration of polyphenolic compounds, including resveratrol, which may be used in the production of grape-derived liquids, such as vinegars. Following the second pressing, the mixture is dried to remove essentially all of the remaining moisture. At the same time, the grape seeds may be separated from the mixture. The mixture may be dried and the grape seeds separated by any suitable means that does not subject the mixture to elevated temperatures that can reduce the concentration or degrade the efficacy of the polyphenolic compounds present in the mixture. For example, the mixture may be dried using high air flow convection without the addition of significant heat (greater than about 120° F. (50° C.)) from an external heat source. The grape seeds may be separated from the mixture during or after drying using a separator that likewise does not introduce significant (greater than about 120° F. (50° C.)) heat from an external heat source. The separated grape seeds may then be processed in a conventional manner to produce a dry powder, such as a relatively pure grape seed powder. Alternatively or in addition, the grape seeds may be pressed again under high force to produce a grape seed sludge or oil having a high concentration of a unique profile of polyphenolic compounds, including resveratrol. Regardless, the re-pressed and dried mixture is then ground in any conventional manner to produce a dry, grape-derived powder product for use alone or in combination with other constituents as a food additive, nutriment, dietary supplement, nutraceutical, pharmaceutical or cosmetic product, such as an over-the-counter health or beauty aid, having a high concentration of a unique profile of polyphenolic compounds, including resveratrol. Both of the pressing steps, the drying step and the grinding step are performed at a temperature that is at or below ambient temperature, for example less than about 77° F. (25° C.), and preferably between about 32° F. (0° C.) and 60° F. (15° C.), in order to promote the further production of, as well as to preserve the efficacy of, the polyphenolic compounds, including resveratrol.

In a particular preferred embodiment, the organic plant matter consists of a mixture of grapes and rice hulls. While any known variety of grapes can potentially produce a dry powder comprising a beneficial bioactive component, the Muscadine variety of grape is preferred due to its inherently higher concentration of polyphenolic compounds, and especially resveratrol. Accordingly, the organic plant matter in this embodiment consists of a mixture of Muscadine grapes and rice hulls. An enzyme may optionally be added to the mixture for the purposes previously described. Rice hulls are preferred over other varieties of press filler components because rice hulls contain beneficial nutrients and fiber. Furthermore, they are rich in B vitamins and contain an active form of vitamin E, which acts synergistically with the polyphenolic compounds in the grapes and grape by-products, and serves to further protect the polyphenolic compounds from oxidative degradation. The ratio of Muscadine grapes to rice hulls by weight is at least about 10:1 and does not exceed about 50:1. More preferably, the ratio of Muscadine grapes to rice hulls is between about 20:1 by weight and about 30:1 by weight. In a particular example, about 100 pounds of rice hulls are mixed with about 1 ton (2200 pounds) of Muscadine grapes. The juice of the Muscadine grapes is extracted from the mixture in any suitable manner, such as by a conventional bladder press or screw press, to produce Muscadine grape juice having a high concentration of polyphenolic compounds, including resveratrol. The grape juice may be used as a constituent of a food product, such as a juice or juice by-product, or may be fermented in a suitable manner to produce wines.

The residue after removing the bulk of the moisture from the mixture consists of Muscadine grape pomace or marc, Muscadine grape seeds and rice hulls. The residue is placed in cold storage for a suitable period of time as described above and thereafter re-pressed to extract essentially all of the remaining moisture, which consists primarily of Muscadine grape juice having an exceptionally high concentration of polyphenolic compounds, including resveratrol. Preferably, the Muscadine grape juice is collected and processed into a juice product for use alone or in combination with other constituents as a food additive, nutriment, dietary supplement, nutraceutical, pharmaceutical or a component of a cosmetic product. The residue is then dried and the grape seeds separated from the pressed Muscadine grape pomace or marc and rice hulls in the manner previously described. Alternatively, the grape seeds may be separated from the grape pomace or marc and rice hulls utilizing a conventional macerator and separation process. However, it is desirable that the grape seeds are separated from the residue without the addition of water or any liquid solvent that might dilute the residue and possibly wash out a portion of the beneficial polyphenolic compounds, including resveratrol. The grape seeds may be processed by further pressing, drying or grinding, preferably at high pressure and low temperature, to produce a dry powder, sludge or oil product, such as a relatively pure Muscadine grape seed powder or grape seed oil having an enhanced concentration and unique profile of polyphenolic compounds, including resveratrol. The residual grape seed cake is essentially fat-free and likewise may be ground into a fat-free grape seed powder having an enhanced concentration and unique profile of polyphenolic compounds, including resveratrol.

Although optional, processing the Muscadine grapes with the addition of an active enzyme softens the skins of the grapes, thereby facilitating the pressing, re-pressing, separating and grinding processes, as well as promoting and preserving the production of polyphenolic compounds, thus ultimately resulting in a higher concentration of free resveratrol available for absorption from the dry powder. It is believed that in addition to the nutritional benefits previously mentioned, the rice hulls serve to prevent the mixture from congealing during and after pressing, for example by squeezing the mixture in a conventional bladder press or screw press. Most importantly, the mixture of Muscadine grapes and rice hulls (along with the optional active enzyme) is stored for an extended period of time at a temperature that is sufficiently below room temperature, and preferably is only slightly above freezing, in order both to promote the continued production of polyphenolic compounds and to preserve the already existing or metabolized polyphenolic compounds. In particular, storing the grape pomace or marc and rice hulls after pressing has been proven to stimulate the continued production of polyphenolic compounds, including resveratrol. Meanwhile, processing the mixture of Muscadine grapes and rice hulls at a temperature that does not exceed about 60° F. (15° C.) preserves the polyphenolic compounds, including resveratrol already present in the mixture or produced during cold storage. It is believed that the reduced temperature effects a higher concentration of the polyphenols by stressing the constituents and thereby steering the remaining metabolic synthesis “machinery” of the grapes and rice hulls along the path of producing more polyphenolic compounds, including resveratrol.

Preferably, the pressed residue is stored at a temperature only slightly above 32° F. (0° C.) in order to promote continued metabolic synthesis, thereby increasing the resulting concentration of the polyphenolic compounds in the resulting dry powder, as well as the grape seeds separated from the residue and the corresponding products derived from the grape seeds. The mixture and pressed residue is processed at a temperature between about 32° F. (0° C.) and 60° F. (15° C.) in order to preserve the efficacy of the polyphenolic compounds, including resveratrol, by not subjecting the polyphenolic compounds to damaging elevated temperature. The pressed residue is stored at the reduced temperature for an extended period of time, preferably between at least about 14 days and 6 months (180 days), to optimize the efficacy of the nutritional, health and/or therapeutic benefit provided by the polyphenolic compounds, including resveratrol. Typically, the pressed residue is stored at the reduced temperature for between 1 month (30 days) and about 3 months (90 days). During the extended period of time, a portion of the remaining moisture content of the pressed residue is removed due to normal evaporation. Thereafter, the pressed residue is preferably pressed again as previously described and then further dried using a suitable low heat/high air flow process.

In this preferred embodiment of the invention, the pressed residue is preferably dried using a conventional heat source accompanied by air convection at a temperature not exceeding about 120° F. (50° C.). Finally, the cold-processed, dried residue is separated into essentially three components consisting of: 1) grape pomace or marc comprising predominantly grape skins (husks); 2) a lesser consistency mixture comprising a combination of about sixty percent (60%) grape skins and about forty percent (40%) rice hulls by weight; and 3) grape seeds. The grape skins are ground in any conventional manner to produce a dry powder derived principally from Muscadine grapes for use alone or in combination with other constituents as a food additive, nutriment, dietary supplement, nutraceutical, pharmaceutical or cosmetic product, such as an over-the-counter health or beauty aid, having a high concentration of polyphenolic compounds, including resveratrol. The separated grape seeds are further pressed, for example by a cold press, to extract relatively pure grape seed oil and a high residue oil sludge forming a dry grape seed meal cake which is essentially devoid of oil. The grape seed meal cake processed in this manner is high in fiber, low in fat (oil free), low in carbohydrate and contains a relatively high concentration of naturally occurring antioxidants, such as resveratrol. Similarly, the oil processed in this manner has an exceptionally high concentration of polyphenolic compounds, including resveratrol. The oil has had minimal exposure to heat, and therefore, has experienced minimal degradation or oxidation. In addition, the extended period of time in cold storage has enabled more beneficial polyphenolic compounds to be produced in the grape seeds and the resulting grape seed oil. Alternatively, the grape seeds may be dried and ground in the same manner as the pressed residue into a dry grape seed powder that is high in fiber, low in fat (i.e., oil free), low in carbohydrate and contains a relatively high concentration of naturally occurring antioxidants, including resveratrol. In yet another embodiment, the grape seeds may be further dried, for example by passing air around the seeds at high velocity and at a mildly elevated temperature, and then ground or fragmented in any suitable manner to produce a dry grape seed powder that is high in fiber, low in carbohydrate and contains a relatively high concentration of naturally occurring antioxidants, including resveratrol.

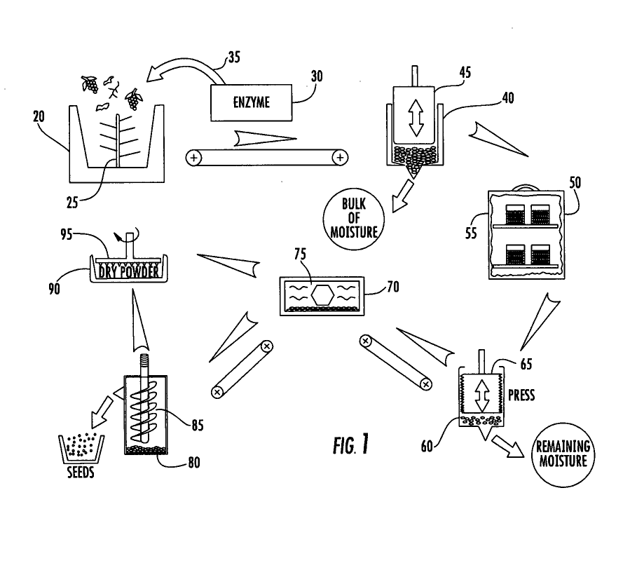

Referring now to the accompanying drawings, FIG. 1 shows an exemplary apparatus, indicated generally at 10, for processing organic plant matter into a dry powder, oil or juice product according to the present invention. The apparatus 10 comprises a mixing station 20, an optional enzyme introducing station 30, a first pressing (i.e., de-juicing) station 40, a cold storage station 50, a second pressing station 60, a drying station 70, an optional seed separating station 80, and a grinding/fragmenting station 90. One or more of the mixing station 20, the enzyme introducing station 30, the cold storage station 50, the second pressing station 60, the drying station 70 and the seed separating station 80 may be utilized in different combinations and in the same or different order in various embodiments of the processing methods intended to be encompassed by the present invention. For example, in the broadest embodiment of the methods it is envisioned that organic plant matter consisting essentially of Muscadine grapes and rice hulls will be mixed together at the mixing station 20, pressed at the first pressing station 40 to extract the bulk of the moisture (i.e., grape juice) from the mixture, and the resulting grape pomace or marc and rice hulls subsequently ground at the grinding/fragmenting station 90 to produce a dry powder derived from Muscadine grapes having a high concentration of polyphenolic compounds, including resveratrol.

In another contemplated embodiment of a method according to the invention grapes and grape by-products are mixed together at the mixing station 20 and an active enzyme is added to the mixture at the enzyme introducing station 30 to soften the skins of the grapes. The mixture, including the active enzyme, is then returned to the mixing station 20 where rice hulls are added to the mixture in a predetermined ratio of grapes to rice hulls. The mixture is then pressed at the first pressing station 40 to extract the bulk of the moisture and the residue is stored at a temperature slightly above freezing for an extended period of time at the cold storage station 50. After the extended period in cold storage is concluded, the residue is pressed again at the second pressing station 60 to extract essentially all of the moisture remaining in the residue. The moisture is collected and further processed for use in other products, and the re-pressed residue is dried at the drying station 70. At the seed separating station 80, the grape seeds are separated from the re-pressed and dried residue and the grape seeds are further processed for use in other products. Finally, the re-pressed, dried and separated residue is ground at the grinding/fragmenting station into a fine, dry powder having a high concentration of polyphenolic compounds, including resveratrol.

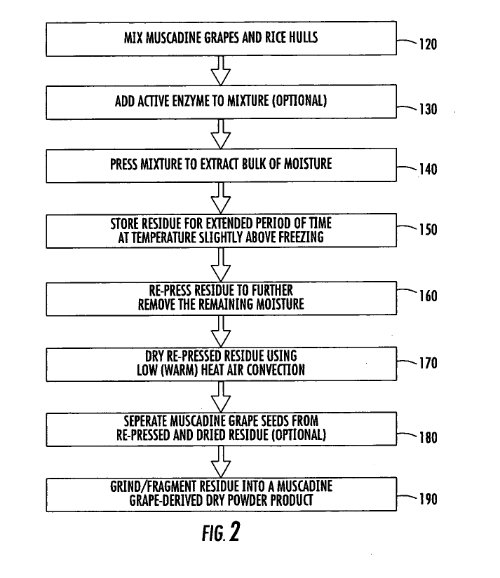

FIG. 2 is a flowchart illustrating the steps of an exemplary method 110 utilizing the apparatus of FIG. 1 to process Muscadine grapes and rice hulls into a dry powder according to the present invention. The method 110 comprises a first step 120 of mixing Muscadine grapes and rice hulls in a predetermined proportion. In an optional second step 130, an active enzyme is added to the mixture of Muscadine grapes and rice hulls. The active enzyme is introduced into the mixture of Muscadine grapes and rice hull to soften the grape skins (husks), and thereby facilitate the break down of the mixture and the later maceration and powdering process, as well as to promote the metabolic synthesis of polyphenolic compounds, including resveratrol. In a third step 140, the mixture is pressed to extract the bulk of the grape juice present in the Muscadine grapes. The juice is collected and further processed for use in a Muscadine grape-derived juice product having a high concentration of polyphenolic compounds, including resveratrol. Pressing the mixture also results in a residue of Muscadine grape pomace or marc, active enzyme and rice hulls that includes Muscadine grape seeds. The method 110 comprises a fourth step 150 of storing the residue for an extended period of time at a temperature only slightly above 32° F. (0° C.) in order to stimulate and promote the continued metabolic synthesis of polyphenolic compounds, including resveratrol, in the residue. The method 110 comprises a fifth step 160 of re-pressing the residue to remove essentially all of the moisture remaining in the residue following the cold storage period. The method 110 comprises a sixth step 170 of drying the re-pressed residue using a heat source accompanied by warm air convection at a temperature not exceeding about 120° F. (50° C.). The method 110 comprises an optional seventh step 180 of separating the Muscadine grape seeds from the residue. The method 110 as described herein comprises a final eighth step 190 wherein the pressed, cold-stored, re-pressed and dried residue is ground or fragmented into a dry powder derived from Muscadine grapes for use alone or in combination with other constituents as a food additive, nutriment, dietary supplement, nutraceutical, pharmaceutical or cosmetic product, such as an over-the-counter health or beauty aid, having a high concentration of polyphenolic compounds, including resveratrol. The foregoing processing steps (except the drying step 170) are preferably performed at a temperature not exceeding about 60 F (15 C) in order to preserve the relatively high concentration of polyphenolic compounds, including resveratrol, present, promoted or preserved in the residue.

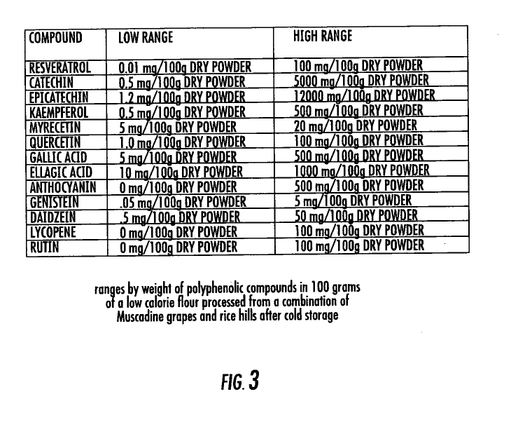

A dry powder derived principally from Muscadine grapes and rice hulls was produced according to the manner shown and described herein and is commercially available from Muscadine Naturals of Lewisville, N.C. FIG. 3 is a table listing the available ranges by weight of various polyphenolic compounds per 100 grams (100 g) of the dry powder from the mixture of Muscadine grapes and rice hulls according to the method of FIG. 2 using the apparatus of FIG. 1. The dry powder processed according to the methods described herein and containing one or more of the polyphenolic compounds listed in the table of FIG. 3 have been observed to provide nutritional, health and/or therapeutic benefits. In particular, case studies have shown the polyphenolic compounds present in the dry powder lower total cholesterol. Furthermore, the antioxidants, especially resveratrol, present in the dry powder have been shown to eliminate free radicals, and therefore, are beneficial in the prevention of heart disease. An unexpected benefit though was discovered after a number of male and female individuals had used the dry powder for a period of time. Users of the dry powder, whether in gelatin capsule form or in the form of a powder mixed with a liquid, reported an increased interest in sexual activity. Initially, the increased sexual desire, or libido, was attributed to the improvement in overall general health that accompanies regular and repeated use of the dry powder. However, as the number of users of the dry powder continued to grow, more and more users admitted to experiencing dreams of an erotic nature and an increased sexual desire. RD, a 70 year old male has been on the dry powder for over a year. He has regular erotic dreams and a strong interest in sex. This was not the case before he started using the dry powder regularly. Among male users 40 years of age and older, there are consistent reports that sexual performance improved along with enhanced libido. The frequency of increased sexual desire and erotic dreams was most prevalent among older males, but was apparent among users of all ages and both sexes. In a particular case study, a 62 year old male (RK) had suffered a stroke rendering him weak on the left side of his body and unable to care for himself. Prior to his stroke RK had little interest in sex. He started on the dry powder, taking 2 capsules twice a day. In just 6 weeks his 35 year old caretaker was pregnant with his child. RK admitted to having more of an interest in sex and erotic dreams about 3 weeks after he started taking the powder. In another case study, a 42 year old male banker (GT) and his 39 year old wife (LT) both started on the dry powder and about 3 to 4 weeks after doing so noticed a greater interest in sex manifested by more erotic dreams during sleep along with a greater frequency of sexual activity with each other. In yet another case study, a 64 year old male (WM) was newly married to a 45 year old woman. Prior to their marriage and honeymoon, WM had an infrequent interest in sex. About 3 weeks prior to the marriage and honeymoon, WM started on the dry powder (2 capsules twice daily), as did his wife. Both noticed an increased interest in sex with increased erotic dreams at night and an enhanced quality and frequency of sexual encounters with each other.

After a statistically significant number of users reported the same benefits, a study was undertaken to determine whether the dry powder provides relief from sexual dysfunction in the same manner as common ED drugs, such as Viagra®, Levitra® and Cialis®. A review of current publications relating to resveratrol and polyphenols revealed striking similarity with the physiological mechanism of the common ED drugs previously discussed herein. For example, a publication from the Department of Pharmacological Sciences, Faculty of Pharmacy, at the University of Milan in Milan, Italy, agrees that a moderate consumption of red wine may reduce the risk of cardiovascular diseases via wine-derived phenolic compounds. The publication notes that a variety of biological mechanisms have been proposed for the action of wine-derived phenolic compounds, including nitric oxide (NO) mediated vasorelaxation. Accordingly, the study examined whether the vasodilating effect of wine-derived phenolic compounds is associated with the inhibition of type 5 phosphodiesterase (PDE-5). The study determined that red wine and the extracts from the grape skin inhibited PDE-5 activity and concluded that polyphenols-induced vasorelaxation may also be sustained by smooth muscle PDE inhibition resulting from anthocyanins present in red wines and grapes. (See In vitro inhibition of human cGMP-specific phosphodiesterase-5 by polyphenols from red grapes; Dell'Agli M., Galli G.V., Vrhovsek U., Mattivi F., Bosisio E.). Another publication from the Department of Pharmacology at the Johannes Gutenberg University in Mainz, Germany tested polyphenolic constituents of red wine for their ability to enhance eNOS expression in humans and found trans-resveratrol to be the most efficacious stimulator of eNOS expression (and eNOS transcription). The anthocyanins cyanidin, the hydroxycinnamic acids p-coumaric acid and caffeic acid, and the phenolic acids benzoic acid and vanilla acid also enhanced eNOS expression moderately. Thus, the study concluded that the increase in eNOS in response to red wine involves several polyphenolic compounds with a major contribution from trans-resveratrol and lesser contributions from cinnamic and hydroxycinnamic acids, cyanidin, and some phenolic acids. (See A blend of polyphenolic compounds explains the stimulatory effect of red wine on human endothelial NO synthase; Wallerath T, Li H., Godtel-Ambrust U., Schwarz P. M., Fostermann U.). Yet another publication from the Department of Pharmacology at the University of Milan determined from in vitro studies that the effect of red wine polyphenols on the vascular tone is likely due to both short-term and long-term mechanisms. NO-mediated vasorelaxation represents the short-term response to wine polyphenols, while the long-term properties of polyphenolic compounds increase endothelial NO synthase expression acting on the promoter activity. (See Vascular effects of wine polyphenols; Dell'Agli M., Busciala A., Bosisio E.).

From the above and other publications, the inventor of the present inventions hypothesized that the dry powder commercially available from Muscadine Naturals and processed according to the proprietary methods shown and described in U.S. patent application Ser. No. 11/442,794 provide a dietary supplement that includes resveratrol in an amount sufficient to permit release of nitric oxide (NO) during sexual stimulation and at least one polyphenol in an amount sufficient to inhibit formation of type 5 phosphodiesterase PDE-5). As a result, the dietary supplement produces the same basic physiological mechanism as the common ED drugs that cause release of NO and suppression of PDE-5 (thereby increasing the sensitivity of tissue to NO) in response to sexual stimulation. It is known that resveratrol alone releases NO, but has no recognizable effect on PDE-5. Therefore, it is believed that the high concentration of the resveratrol and the high concentration of the at least one polyphenol in the dry powder produced by the “cold storage and processing” methods combine to provide the “Viagra® like” effects that have been reported by users of the dry powder. Unlike the common ED drugs, the dry powder provides additional health and therapeutic benefits, including lowering cholesterol and possibly preventing heart disease. In addition, the resveratrol and at least one polyphenol apparently accumulate in the metabolic system of the user over a period of time. Thus, the libido enhancing effect of the dry powder is available at any time and sexual activity need not be planned in contrast to the common ED drugs which must be taken within a prescribed time prior to planned sexual activity. Furthermore, the dry powder dietary supplement appears to have the same general libido enhancing effect on both human males and human females.

In a preferred embodiment, the dietary supplement is a dry powder derived from Muscadine grapes that are pressed to remove at least about fifty percent (50%) of the moisture content of the grapes. The pressed grapes (i.e., predominately grape skins) are stored at a temperature greater than about 32° F. (0° C.) and less than about 60° F. (15° C.). The pressed grapes are stored for at least about 14 days, and more preferably, for at least about 3 months (90 days). The pressed and stored grapes are dried and then ground into the dry powder. The pressed and stored grapes are dried using high air flow convection without the addition of heat in excess of about 120° F. (50° C.) and wherein the dried grapes are ground at a temperature less than about 77° F. (25° C.). The fully processed dietary supplement comprises a concentration of the resveratrol that is greater than at least three (3) times the concentration of the resveratrol when the grapes are not stored for at least 14 days at a temperature greater than about 32° F. (0° C.) and less than about 60° F. (15° C.) before the pressed and stored grapes are dried and then ground. The concentration of the resveratrol is preferably between about 0.01 mg and about 100 mg per 100 g of the dry powder. The concentration of the at least one polyphenol is preferably between about 0.04 mg and about 400 mg per 100 g of the dry powder. The dietary supplement is processed in the manner described herein such that the dry powder comprises the resveratrol in a therapeutically effective amount and the at least one polyphenol in a therapeutically effective amount to provide the effect of enhancing human libido. In another preferred embodiment, the dietary supplement further comprises rice hulls mixed with the grapes in a ratio of grapes to rice hulls by weight of at least about 10:1. The dietary supplement may also comprise at least one active enzyme selected from the group consisting of pectinase and amylase types to facilitate breaking down the grape skins during the pressing, drying and grinding steps. Use of the dietary supplement provides a method for enhancing human libido wherein the dry powder comprising a therapeutically effective amount of resveratrol to release nitric oxide (NO) during sexual stimulation and a therapeutically effective amount of a polyphenol to inhibit formation of type 5 phosphodiesterase (PDE-5) is provided. At least about 50 mg of the dry powder is consumed by the user at least about once a day for a period of at least about 21 days.

Various exemplary embodiments of the present invention have been shown and described herein. However, the invention should not be construed as being limited to the particular embodiments shown and described herein. Instead, the invention should be construed broadly to encompass any and all embodiments that are reasonably foreseeable and that fall within the scope of the appended claims.

1. A dietaiy supplement comprising:
resveratrol in an amount sufficient to permit release of nitric oxide (NO) during sexual stimulation; and
at least one polyphenol in an amount sufficient to inhibit formation of type 5 phosphodiesterase (PDE-5).

2. A dietary supplement according to claim 1, wherein the dietary supplement is a dry powder derived from grapes that are pressed to remove at least about fifty percent (50%) of the moisture content of the grapes and the pressed grapes are stored at a temperature greater than about 32° F. (0° C.) and less than about 60° F. (15° C).

3. A dietary supplement according to claim 2, wherein the pressed grapes are stored for at least about 14 days.

4. A dietary supplement according to claim 2, wherein the pressed and stored grapes are dried and then ground into the dry powder.

5. A dietary supplement according to claim 4, wherein the dry powder comprises a concentration of the resveratrol that is greater than at least three (3) times the concentration of the resveratrol when the grapes are not stored for at least 14 days at a temperature greater than about 32° F. (0° C.) and less than about 60° F. (15° C.) before the pressed and stored grapes are dried and then ground.

6. A dietary supplement according to claim 2, wherein the concentration of the resveratrol is between about .01 mg and about 100 mg per 100 g of the dry powder.

7. A dietary supplement according to claim 6, wherein the concentration of the at least one polyphenol is between about .04 mg and about 400 mg per 100 g of the dry powder.

8. A dietary supplement according to claim 4, wherein the pressed and stored grapes are dried using high air flow convection without the addition of heat in excess of about 120° F. (50° C.) and wherein the dried grapes are ground at a temperature less than about 77° F. (25° C).

9. A dietary supplement according to claim 2, wherein the grapes are Muscadine grapes and the pressed grapes consist essentially of Muscadine grape skins.

10. A dietary supplement having the effect of enhancing human libido, the dietary supplement comprising:
resveratrol in a therapeutically effective amount; and
at least one polyphenol in a therapeutically effective amount;
wherein the therapeutically effective amount of resveratrol and the therapeutically effective amount of the at least one polyphenol are derived from grapes that are pressed to remove at least about fifty percent (50%) of the moisture content of the grapes and the pressed grapes are stored at a temperature greater than about 32° F. (0° C.) and less than about 60° F. (15° C).

11. A dietary supplement according to claim 10, further comprising rice hulls mixed with the grapes and wherein the ratio of grapes to rice hulls by weight is at least about 10:1.

12. A dietary supplement according to claim 10, further comprising at least one active enzyme selected from the group consisting of pectinase and amylase.

13. A dietary supplement according to claim 10, wherein the pressed grapes are stored for at least about 14 days.

14. A dietary supplement according to claim 10, wherein the pressed and stored grapes are dried and then ground into a dry powder.

15. A dietary supplement according to claim 14, wherein the dry powder comprises a concentration of the resveratrol that is greater than at least three times the concentration of the resveratrol when the grapes are not stored for at least 14 days at a temperature greater than about 32° F. (0° C.) and less than about 60° F. (15° C.) before the pressed and stored grapes are dried and then ground.

16. A dietary supplement according to claim 14, wherein the concentration of the resveratrol is between about .01 mg and about 100 mg per 100 g of the dry powder.

17. A dietary supplement according to claim 16, wherein the concentration of the at least one polyphenol is between about .04 mg and about 400 mg per 100 g of the dry powder.

18. A dietary supplement according to claim 15, wherein the pressed and stored grapes are dried using high air flow convection without the addition of heat in excess of about 120° F. (50° C.) and wherein the dried grapes are ground at a temperature less than about 77° F. (25° C).

19. A dietary supplement according to claim 10, wherein the grapes are Muscadine grapes and the pressed grapes consist essentially of Muscadine grape skins.

20. A method for enhancing human libido, the method comprising:
providing a dry powder comprising a therapeutically effective amount of resveratrol to release nitric oxide (NO) during sexual stimulation and a therapeutically effective amount of a polyphenol to inhibit formation of type 5 phosphodiesterase (PDE-5); and
consuming at least about 50 mg of the dry powder at least about once a day for a period of at least about 21 days.

21. A method according to claim 20, wherein the dry powder is derived from grapes that are:
pressed to remove at least about filly percent (50%) of the moisture content of the grapes;
stored for at least about 14 days at a temperature greater than about 32° F. (0° C.) and less than about 60° F. (15° C.);
dried to remove essentially all of the moisture content of the grapes; and ground into the dry powder.

22. A method according to claim 21, wherein the pressed and stored grapes are dried using high air flow convection without the addition of heat in excess of about 120° F. (50° C).

23. A method according to claim 21, wherein the grapes are pressed and ground at a temperature less than about 77° F. (25° C).

24. A method according to claim 21, wherein the grapes are mixed with rice hulls in a ratio of grapes to rice hulls by weight of at least about 10:1 and wherein at least one active enzyme selected from the group consisting of pectinase and amylase types is added to the mixture.

25. A method according to claim 20, wherein the dry powder comprises a concentration of the resveratrol that is greater than at least three times the concentration of the resveratrol when the grapes are not stored for at least 14 days at a temperature greater than about 32° F. (0° C.) and less than about 60° F. (15° C.) before the pressed and stored grapes are dried and ground.

26. A method according to claim 25, wherein the concentration of the resveratrol is between about .01 mg and about 100 mg per 100 g of the dry powder and wherein the concentration of the at least one polyphenol is between about .04 mg and about 400 mg per 100 g of the dry powder.
